(19) United States
(12) Patent Application Publication
(10) Pub. No.: US 2004/0068322 A1
(43) Pub. Date: Apr. 8, 2004

(54) Reduced-friction artificial joints and components therefor

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/679,833

(22) Filed: Oct. 6, 2003

Related U.S. Application Data
(60) Provisional application No. 60/416,339, filed on Oct. 4, 2002.

(51) Int. Cl. A61F 2/30; A61F 2/38; A61F 2/36

(52) U.S. Cl. 623/23.39; 623/20.23; 623/22.16; 623/18.11; 623/22.45; 623/22.46

(57) ABSTRACT

Wheels, bearings, or other rotating elements are used to decrease friction in artificial joint components associated with knee and hip replacements, artificial ankles, elbows, wrist, finger joints, toe joints, and intervertebral discs. One or more seals may be used to trap debris inside the artificial joint. The seal may surround the periphery of the component(s), and may be used to hold a fluid within the joint. Various fluids or lubricants are applicable to these embodiments, including water or aqueous solutions, triglyceride oil, soybean oil, an inorganic oil (e.g. silicone oil or fluorocarbon), glycerin, ethylene glycol, or other animal, vegetable, synthetic oil, or combinations thereof could be used. The seal could be made of an expandable elastomer such as those used in medical devices for the cardiovascular system.

### REFERENCE TO RELATED APPLICATION

This application claims priority from U.S. Provisional Patent Application Serial No. 60/416,339, filed Oct. 4, 2002, the entire content of which is incorporated herein by reference.

### FIELD OF THE INVENTION

This invention relates generally to prosthetic joint components and, in particular, to reduced-friction artificial joints.

### BACKGROUND OF THE INVENTION

Many spinal conditions, including degenerative disc disease, can be treated by spinal fusion or through artificial disc replacement (ADR). Since spinal fusion eliminates motion across fused segments of the spine, the discs adjacent to the fused level are subjected to increased stress. The increased stress increases the changes of future surgery to treat the degeneration of the discs adjacent to the fusion.

ADRs offer several advantages over spinal fusion, the most important of which is the preservation of spinal motion. One of the most important features of an artificial disc replacement (ADR) is its ability to replicate the kinematics of a natural disc. ADRs that replicate the kinematics of a normal disc are less likely to transfer additional forces above and below the replaced disc. In addition, ADRs with natural kinematics are less likely to stress the facet joints and the annulus fibrosus (AF) at the level of the disc replacement. Replicating the movements of the natural disc also decreases the risk of separation of the ADR from the vertebrae above and below the ADR.

The kinematics of ADRs are governed by the range of motion (ROM), the location of the center of rotation (COR) and the presence (or absence) of a variable center of rotation (VCOR). Generally ROM is limited by the facet joints and the AF. Motion across prior-art prosthetic joints occurs by rotation and sliding between the components. The resultant friction causes surface wear leading to problems well known to orthopedic surgeons (i.e., fracture of polyethylene trays, polyethylene debris, component loosening, etc.). “Revision” surgery is frequently required to correct the problems associated with component wear.

### SUMMARY OF THE INVENTION

This invention is broadly directed to decreasing friction in artificial joint components. In the preferred embodiments, wheels, bearings, or other rotating elements are used for this purpose. Although this disclosure focuses on knee and hip replacements, the invention can also be used for other artificial joints including artificial ankles, elbows, wrist, finger joints, toe joints, and intervertebral discs.

In all of the embodiments, one or more seals may be used to trap debris inside the artificial joint. The seal may surround the periphery of the component(s), and may be used to hold a fluid within the joint. Various fluids or lubricants are applicable to these embodiments, including water or aqueous solutions, triglyceride oil, soybean oil, an inorganic oil (e.g. silicone oil or fluorocarbon), glycerin, ethylene glycol, or other animal, vegetable, synthetic oil, or combinations thereof could be used. The seal could be made of an expandable elastomer such as those used in medical devices for the cardiovascular system.

### BRIEF DESCRIPTION OF THE DRAWINGS

FIG. 1 shows a view of the anterior aspect of an artificial knee of the present invention;

FIG. 2 shows a view of the lateral aspect of the TKR shown in FIG. 1;

FIG. 3 is a coronal cross section of the TKR shown in FIG. 1;

FIG. 4 is a sagittal cross-section of the TKR of FIG. 1;

FIG. 5 is an alternative embodiment illustrating the use of more than one wheel;

FIG. 6 shows the end of a patella with an attached patella resurfacing component;

FIG. 7 is a view of the articular aspect of the prosthetic patellar component shown in FIG. 6;

FIG. 8 is a coronal cross section of an alternative embodiment of the invention, wherein the wheel is housed within the tibial component of the TKR;

FIG. 9 is a sagittal cross section of a hip embodiment of the invention;

FIG. 10 is an exploded view of the embodiment of FIG. 9;

FIG. 11A is a view of the lateral aspect of the rotating shaft of the present invention;

FIG. 11B is a view of the lateral aspect of an alternative embodiment of the rotating shaft;

FIG. 12 is a view of the lateral aspect of a TKR according to the invention with optional cables or bands;

FIG. 13 shows a preferred embodiment including a separately disclosed shock absorber in the intramedullary portion of the femoral component;

FIG. 14 is an exploded view of the neck-bearing-head-c-clip arrangement;

FIG. 15 is an alternative embodiment of FIG. 9 with roller bearings; and

FIG. 16 is a sagittal cross section of FIG. 9 with roller bearings.

### DETAILED DESCRIPTION OF THE INVENTION

Although this disclosure focuses on knee and hip replacements, it will be appreciated by those of skill that the invention can also be used for other artificial joints including artificial ankles, elbows, wrist, finger joints, toe joints, and intervertebral discs.

Knee Embodiments

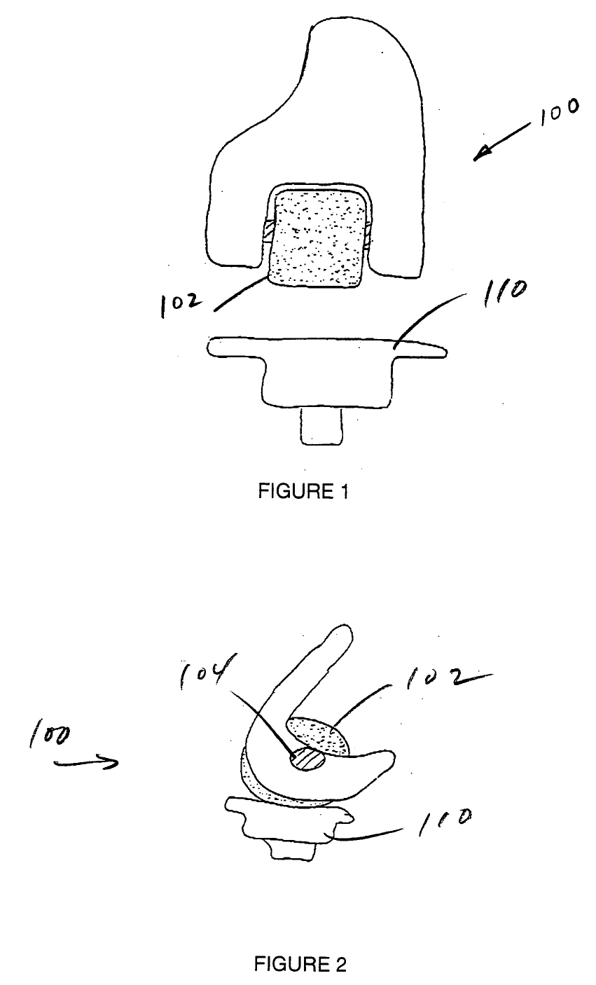

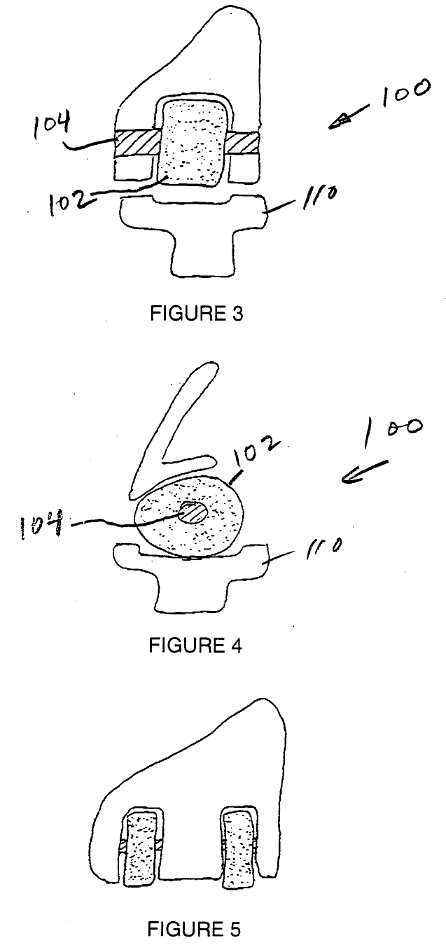

FIG. 1 is a view of the anterior aspect of a total knee replacement (TKR) according to the invention shown generally at 100 including a wheel 102 rotatable about an axle 104. FIG. 2 is a view of the lateral aspect of the TKR drawn in FIG. 1. FIG. 3 is a coronal cross section of the TKR drawn in FIG. 1. Although not shown, the tibial component 110 may have a recess to cooperate with the wheel of the femoral component. FIG. 4 is a sagittal cross-section of the TKR of FIG. 1. FIG. 5 is an alternative embodiment illustrating the use of more than one wheel.

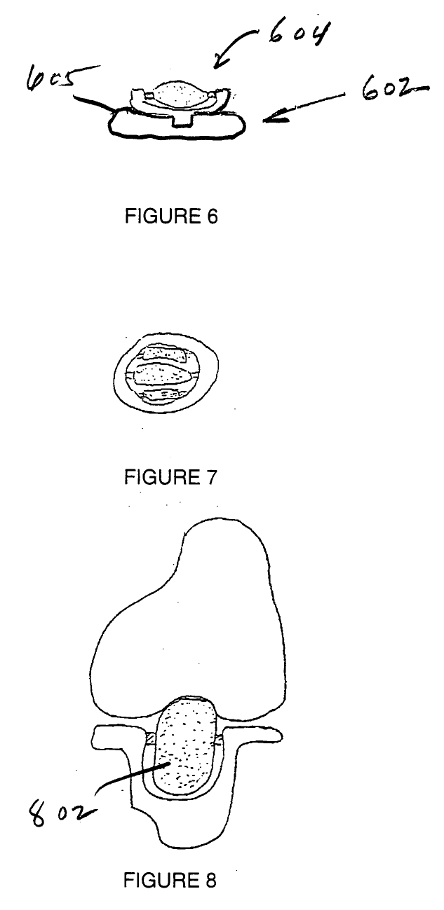

The embodiment of FIG. 6 shows the end of a patella 602 with an attached patella resurfacing component 604. The area 605 represents the bone of the patella. FIG. 7 is a view of the articular aspect of the prosthetic patellar component drawn in FIG. 6. FIG. 8 is a coronal cross section of an alternative embodiment of the invention, including a wheel 802 housed within the tibial component of a TKR.

FIG. 12 is a view of the lateral aspect of a TKR according to the invention with optional cables or bands 1202 to prevent the femoral and tibial components from dislocating.

Hip Embodiments

The embodiments of the invention associated with the hip reduce friction across the femoral-acetabular articular surface. Rotation about two different planes, preferably at ninety degrees to one another, also reduces the chances of dislocation.

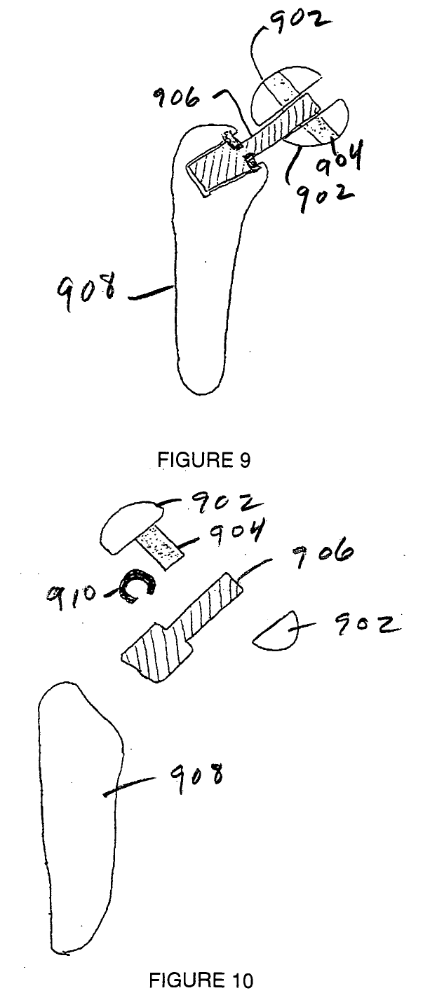

FIG. 9 is a sagittal cross section of a hip embodiment of the invention. FIG. 10 is an exploded view. Wheels 902 rotate about an axle 904. A shaft 906 (which may rotate) connects the first axle to the portion of the femoral component 908 that fits within the medullary canal. A removable c-ring 910 holds the rotating shaft in the intramedullary component.

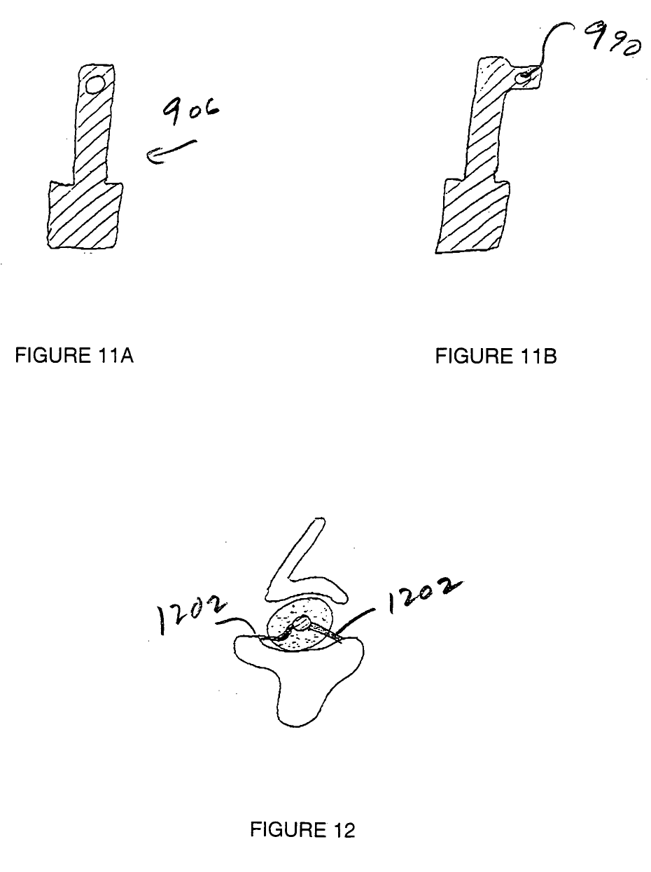

FIG. 11 A is a view of the lateral aspect of the rotating shaft. The hole for the axle is centrally located in this embodiment. FIG. 11B is a view of the lateral aspect of an alternative embodiment of the rotating shaft including an offset hole 990.

Balls bearings could also surround the rotating axle and/or shaft. The bearings could be sealed, with an optional membrane being used to surround the assembly to seal the moveable components from the body. For example, such a seal could extend from the acetabular component to the intramedullary portion of the femoral component. A lubricating fluid may be placed between the membrane and the movable components. Fluid from the hip joint could naturally lubricate the bearings.

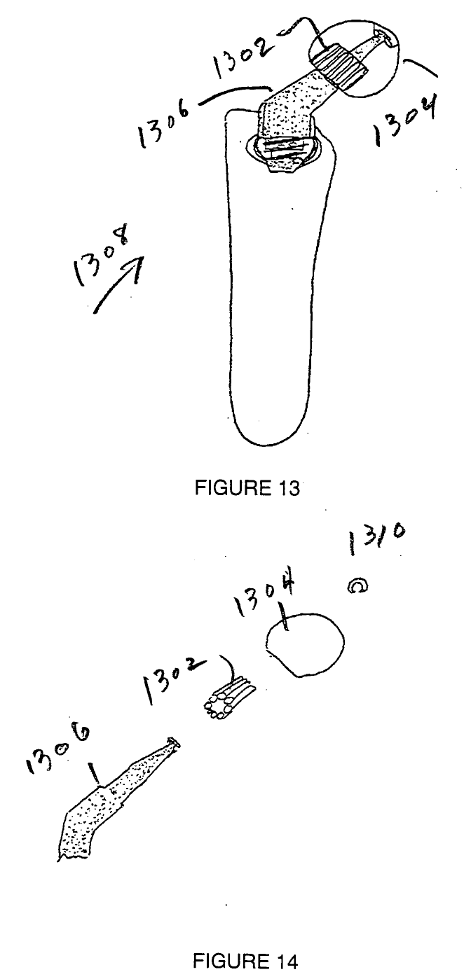

FIG. 13 depicts an embodiment of the invention including a separately disclosed shock absorber in the intramedullary portion of the femoral component. The head of the component rides on needle like roller bearings 1302 between the head 1304 and the neck 1306 of the prosthesis 1308. The head is attached to the neck with a c-clip 1301. FIG. 14 is an exploded view of the neck-bearing-head-c-clip arrangement. The bearings could be cylinder shaped as shown if the neck is cylindrical. Alternatively, the bearings could be trapezoidal shape if the neck is tapered.

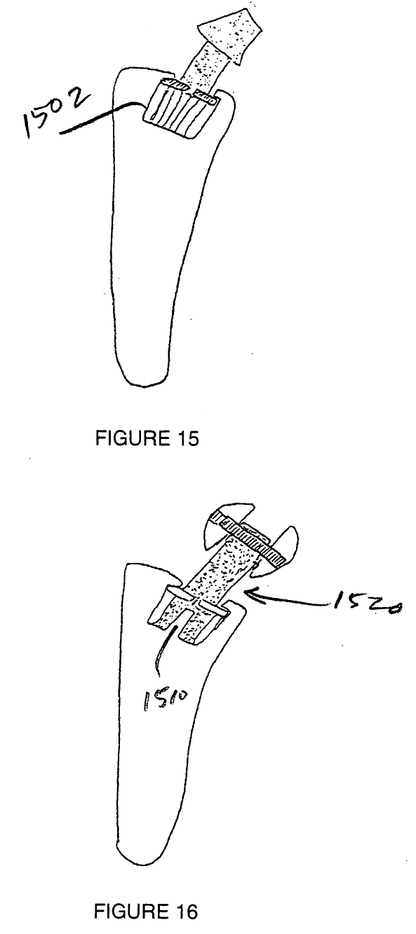

FIG. 15 is an alternative embodiment of FIG. 9 with roller bearings 1502. FIG. 16 is a sagittal cross section of FIG. 9 with roller bearings. A projection 1510 from the intramedullary portion of the femoral stem could extend into the rotating neck 1520 to help resist the tilling forces between the neck and the femoral stem.

I claim:

1. An artificial joint configured for use between first and second articulating bones, comprising:
a first prosthetic component adapted for coupling to the first bone;
a second prosthetic component adapted for coupling to the second bone; and
a wheel, bearing or other rotating element between the two components to reduce friction during articulation.

2. The artificial joint of claim 1, wherein:
the first prosthetic component is adapted for coupling to a distal femur;
the second prosthetic component is adapted for coupling to a proximal tibia; and
the first prosthetic component includes a roller rotatable about an axis oriented generally medial to lateral.

3. The artificial joint of claim 1, wherein the second prosthetic component includes a recess to receive the roller.

4. The artificial joint of claim 1, including a plurality of rollers, each rotatable about an axis oriented generally medial to lateral.

5. The artificial joint of claim 1, including a debris-containing seal around the first prosthetic component, first prosthetic component, wheel, bearing or other rotating element.

6. The artificial joint of claim 1, including a fluid-containing seal around the first prosthetic component, second prosthetic component, wheel, bearing or other rotating element.

7. The artificial joint of claim 1, wherein the first prosthetic component, second prosthetic component, wheel, bearing or other rotating element are tethered to one or both of the first and second bones.

8. The artificial joint of claim 1, wherein the first prosthetic component, second prosthetic component, wheel, bearing or other rotating element are associated with a patellar replacement.

9. The artificial joint of claim 1, wherein:
the first prosthetic component is adapted for coupling to a proximal femur;
the second prosthetic component is an acetabular component; and
the first prosthetic component includes a rotatable element adapted to engage with the acetabular component.

10. The artificial joint of claim 1, wherein:
the first prosthetic component is adapted for coupling to a proximal femur;
the second prosthetic component is an acetabular component; and
the first prosthetic component includes an element rotatable about two different planes.

11. The artificial joint of claim 10, wherein the two planes are orthogonal to one another.

12. The artificial joint of claim 10, wherein:
the first prosthetic component includes an intramedullary stem; and
the stem rotates about one of the planes.

13. The artificial joint of claim 10, wherein:
the first prosthetic component includes an intramedullary stem; and
the stem includes a shock absorber.
